(19) United States
(12) Patent Application Publication
(10) Pub. No.: US 2026/0260273 A1
(43) Pub. Date: Sep. 3, 2026

(54) SYSTEMS AND METHODS FOR IMPROVING THERAPEUTIC INTERVENTIONS UTILIZING E-COMMERCE PLATFORMS

(71) Applicants: Grzegorz Bulaj, Salt Lake City, UT (US); Dorothy Huntsman, Salt Lake City, UT (US)

(72) Inventors: Grzegorz Bulaj, Salt Lake City, UT (US); Dorothy Huntsman, Salt Lake City, UT (US)

(21) Appl. No.: 19/427,757

(22) Filed: Dec. 19, 2025

Related U.S. Application Data
(60) Provisional application No. 63/737,419, filed on Dec. 20, 2024.

(51) Int. Cl. G06Q 30/0601; G06F 30/13; G16H 10/60; G16H 20/00

(52) U.S. Cl. CPC G06Q 30/0601; G16H 10/60; G16H 20/00; G06F 30/13

(57) ABSTRACT

The invention is a method for improving outcomes of medical interventions by using an integrated e-commerce platform, where the integrated e-commerce platform comprises databases such as an e-commerce platform, a health knowledge database, a household goods database, a consumer goods database, a consumer reports database, an application programming interface, and consumer interfaces. The databases are used to create a therapeutic home environment where the therapeutic home environment is based on biophilic design, environmental quality, and self-care.

### CROSS REFERENCE TO RELATED APPLICATIONS

This application claims priority to copending U.S. Application, Ser. No. 63/737,419, filed on Dec. 20, 2024, which is hereby incorporated by reference for all purposes.

### BACKGROUND

The present disclosure relates generally to improving outcomes of medical interventions by integrating e-commerce platforms with biophilic design principles, evidence-based self-care practices, and consumer education. In particular, applications for e-commerce platforms targeting the home environment as a means to improve treatment and prevention outcomes for chronic diseases are described.

There is a vast need to improve prevention and treatment outcomes for people living with chronic medical conditions. Each person living with a chronic disease experiences diminished health-related quality of life (HRQoL) due to symptoms that impact daily activities and can also lead to disabilities. Chronic diseases negatively impact patients' families (financial burden), as well as healthcare professionals (burnout) and healthcare systems (increasing healthcare costs can reduce profit margins). In addition to impacting public health, high prevalence and incidence of chronic diseases contribute to increasing healthcare spending in the U.S

Prevention and treatment of non-communicable chronic diseases (NCDs) pose multiple challenges to healthcare professionals and systems. A combination of a patient's lifestyle behaviors and DNA polymorphism (genetic diversity) impacts the effectiveness of pharmacological interventions. Adverse effects of prescription drugs, medical errors, healthcare accessibility, and affordability contribute to increased morbidity and mortality. Environmental pollution and commercial determinants of health negatively impact population health.

To reduce the prevalence of chronic diseases, some possible solutions include: (1) early detection, (2) scaling up lifestyle medicine through digital health technologies, and (3) public health interventions through health promotion. These solutions are supported by the growing availability of home-use, in vitro diagnostic tests, advances in artificial intelligence and machine learning (AI/ML), and validation of digital biomarkers that contribute to early detection of medical conditions. Furthermore, digital health technologies enable integration of behavioral interventions and lifestyle medicine with pharmacotherapies.

However, some barriers to mitigate impacts of chronic diseases include: (1) misaligned funding for public health interventions and research on preventing chronic diseases, and (2) inadequate implementation of evidence-based practices to prevent non-communicable chronic diseases in community settings. Despite playing an important role in health and disease, the built environment has been underutilized as a means to improve health outcomes. Thus, there exists a need for systems and methods that improve upon and advance the design of known therapeutic interventions. Examples of new and useful therapeutic interventions relevant to the needs existing in the field are discussed below.

### SUMMARY

The invention is a method for improving outcomes of medical interventions by using an integrated e-commerce platform, where the integrated e-commerce platform comprises databases such as an e-commerce platform, a health knowledge database, a household goods database, a consumer goods database, a consumer reports database, an application programming interface, and consumer interfaces. The databases are used to create therapeutic home environments, promote, and track self-care.

### BRIEF DESCRIPTION OF THE DRAWINGS

FIG. 1 is a perspective view of a health-centric home environment.

FIG. 2 is a perspective view of a comparison of diverse therapeutic targets for the prevention and treatment of chronic diseases.

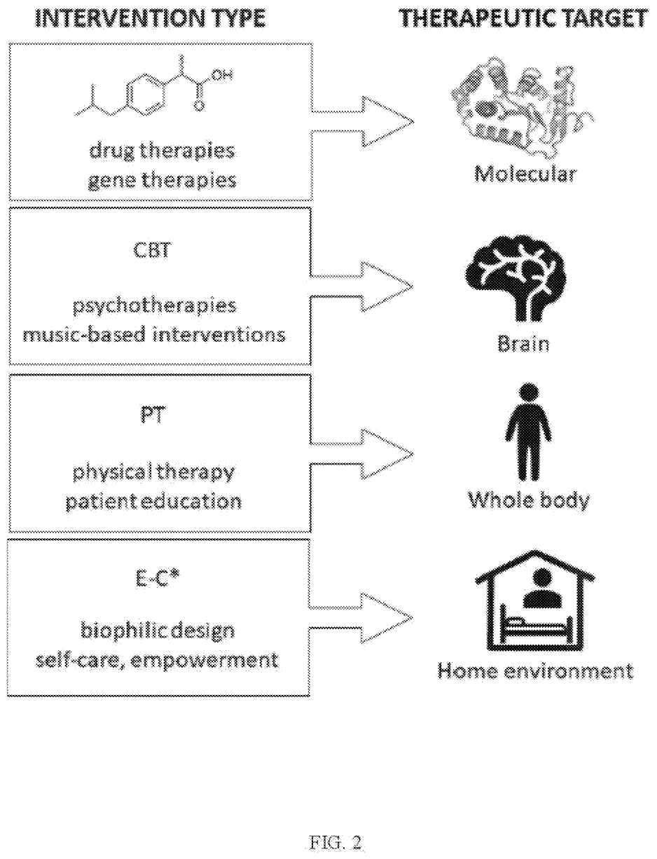

FIG. 3 is a perspective view of biophilic home environments to reduce disease systems.

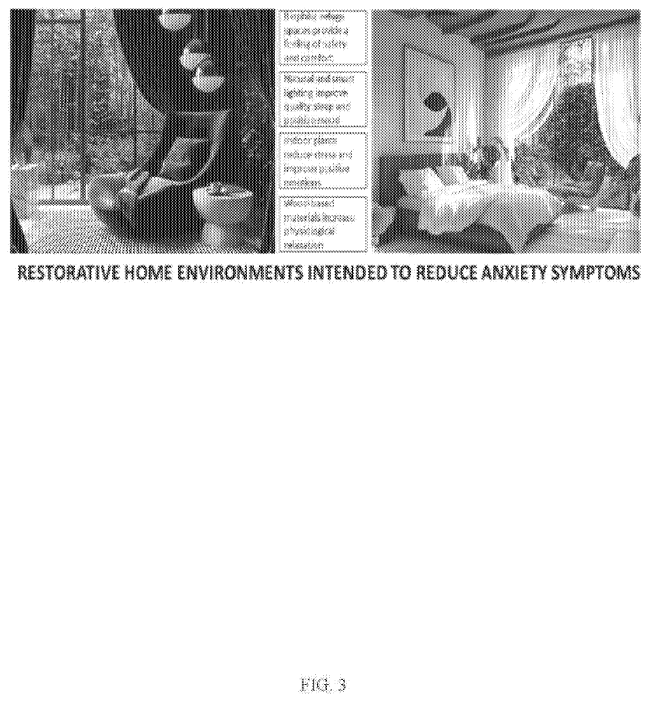

FIG. 4 is a perspective view of a first example of an e-commerce platform to treat and prevent chronic diseases.

FIG. 5 is a block diagram of the e-commerce platform shown in FIG. 4, depicting personalized communications between the health-centric home environment and its occupants.

FIG. 6 is a perspective view of the e-commerce platform shown in FIG. 4 depicting the Application Program Interface.

FIG. 7 is a block diagram of a third example of an e-commerce platform to treat and prevent chronic diseases.

FIG. 8 is a block diagram of the e-commerce platform shown in FIG. 4 in comparison with prior art.

FIG. 9 is a flow chart of the e-commerce platform utilization shown in FIG. 4.

FIG. 10 is a block diagram of the e-commerce platform shown in FIG. 41, depicting a communication application, object recognition, and geo-positioning applications.

FIG. 11 is a flow chart of a second example of an e-commerce platform utilization.

FIG. 12 is a perspective view of the e-commerce platform shown in FIG. 4, depicting a communication application using machine-readable codes.

FIG. 13 is a perspective view of the e-commerce platform shown in FIG. 4, depicting a digital health application integrated with the e-commerce platform.

FIG. 14 is a perspective view of a fourth example of an e-commerce platform utilization.

FIG. 15 is a schematic of the Therapeutic Home Environment relational database.

FIG. 16 is a radar chart illustrating the Therapeutic Home Environment Score.

FIG. 17 is an example of an app for the reduction of migraine symptoms.

FIG. 18 is a user experience flow diagram.

FIG. 19 illustrates converting a household into a therapeutic asset.

FIG. 20 is an example of personalized, multimodal care for migraines.

### DETAILED DESCRIPTION

The disclosed e-commerce platforms will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of e-commerce platform examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls the operation of another electronic device.

### Contextual Details

Ancillary features relevant to the e-commerce platforms described herein will first be described to provide context and to aid in discussing the e-commerce platforms.

### Health-Centric Home Environment

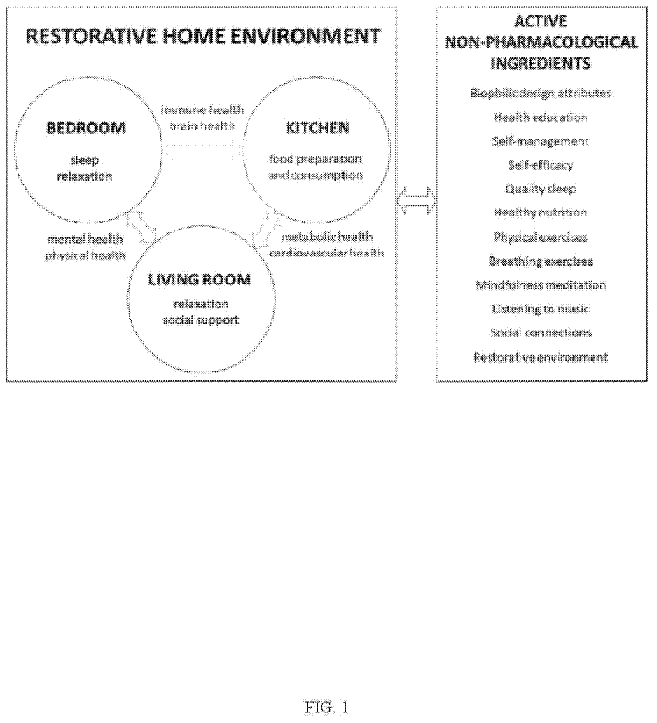

Home environment is defined here as a housing unit comprising physical, chemical, and emotional environments and its occupants who share daily activities and experiences. Housing-health relationships are well characterized, including prospective interventional studies on how internal housing conditions can improve health outcomes. Smart home technologies further illustrate opportunities to create at-home health care applications. Despite playing an important role in health and disease, the built environment has been underutilized as a means to improve health outcomes. The present invention utilizes the inventors' work depicting the biophilic design of the home environment, fostering disease-specific self-care for people living with migraine, chronic pain, and depression. Examples of home-health connections and their relationships with self-care are illustrated in FIG. 1.

There is growing research evidence that diverse non-pharmacological modalities and self-care practices elicit clinically meaningful benefits and can reduce risks for chronic diseases. Therapeutic effects of experiencing nature, physical activities, proper nutrition, quality sleep, listening to music, or social connections offer a means to improve mental and physical health. However, integrating these modalities as “active non-pharmacological ingredients”, as shown in FIG. 1, into medical treatments poses multiple challenges ranging from technical feasibility and clinical validation to regulatory requirements and implementation by healthcare.

### Systems and Methods for Improving Therapeutic Interventions Utilizing E-Commerce Platforms

With reference to the figures, novel Systems and Methods for Improving Therapeutic Interventions Utilizing E-Commerce Platforms will now be described. The novel e-commerce platforms discussed herein function to employ the home environment as a therapeutic target for prevention and adjunct treatment of non-communicable chronic diseases, deploy an e-commerce platform as a delivery system for biophilic interventions, elicit evidence-based self-care and health-centric consumer empowerment, and transform an e-commerce platform into digital health interventions that can be further integrated with pharmacotherapies.

The e-commerce platforms described herein further bridge consumer spending with improving healthcare outcomes by: (1) recognizing home environment as a therapeutic target for the primary, secondary, and tertiary prevention and treatment of chronic diseases, and (2) transforming household goods e-commerce platforms into digital health interventions. The present invention discloses an e-commerce platform that provides restorative and enriched environments grounded in biophilic design and coupled with continuous health education, fostering evidence-based self-care practices and behavior change. The present invention also discloses integration of household goods e-commerce with digital health technologies (software as a medical device, SaMD, prescription drug use-related software, PDURS) to expand health-centric consumer spending through at-home adjunctive therapeutic interventions and precision prevention of chronic diseases.

The reader will appreciate from the figures and description below that the presently disclosed e-commerce platforms address many of the shortcomings of conventional therapeutic interventions and e-commerce platforms. Chronic diseases create diverse problems in medicine and healthcare systems due to the limited effectiveness of pharmaceutical drugs and biologics, lifestyle and environmental factors, as well as competition with industry-driven commercial determinants of health. For example, integrating these modalities as “active non-pharmacological ingredients”, as shown in FIG. 1, into medical treatments poses multiple challenges ranging from technical feasibility and clinical validation to regulatory requirements and implementation by healthcare.

### E-Commerce Platform Embodiment One

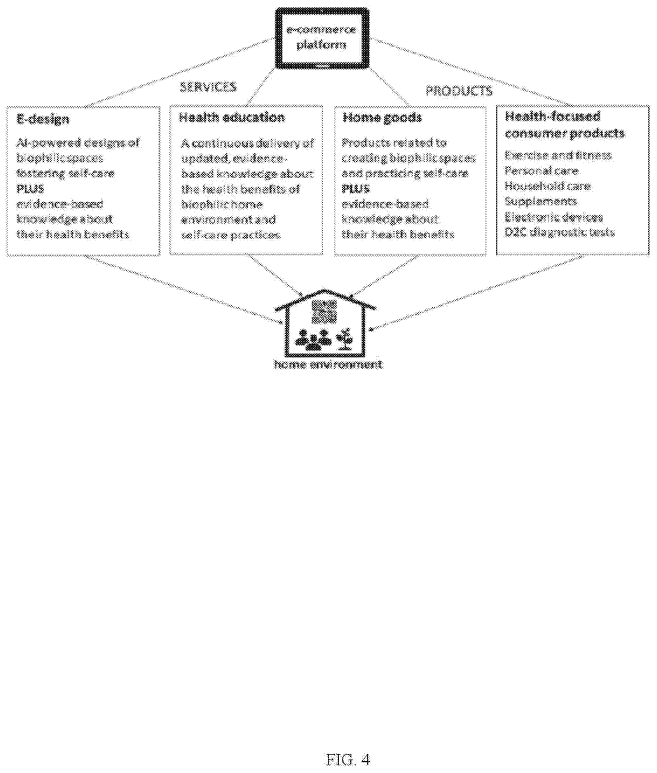

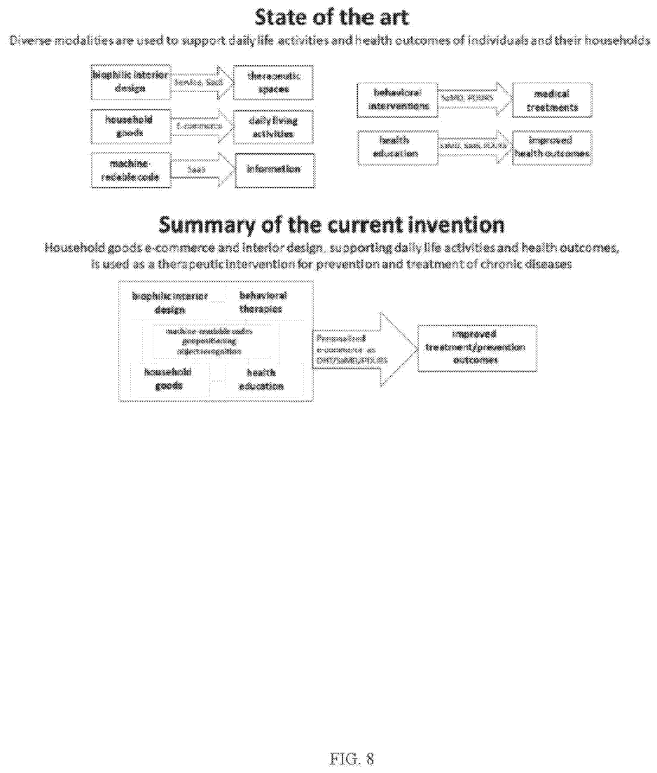

With reference to FIGS. 4-13, a first example of an e-commerce platform, e-commerce platform 100, will now be described. E-commerce platform 100 includes a health knowledge database, a household goods database, a consumer goods database, a consumer reports database, an application programming interface, and consumer interfaces.

### Health Knowledge Database

The health knowledge database functions to store health-focused interior designs and education to support transforming a consumer's home into a restorative environment that provides therapeutic benefits.

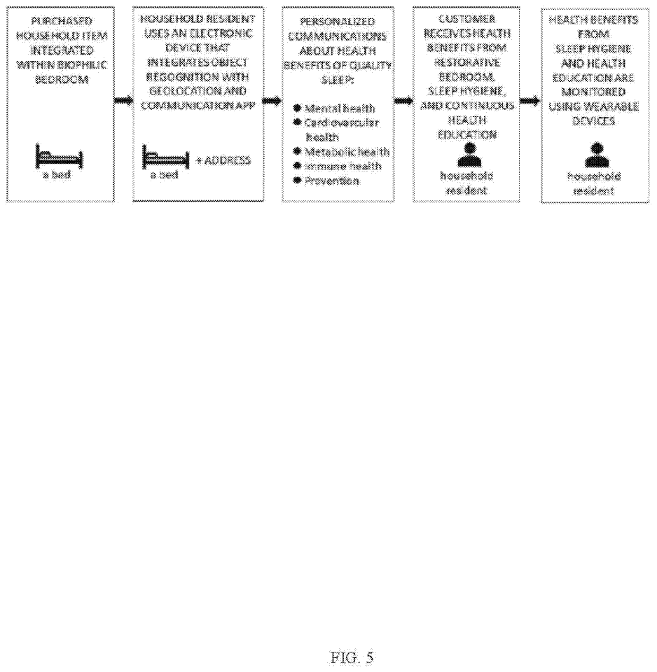

As can be seen in FIGS. 4-6 and 10, the health knowledge database may include associations between household goods and evidence-based health benefits using these household goods. These associations may include the direct or indirect health benefits of a household good or consumer good based on research studies published in peer-reviewed journals. For example, the database may include associations between a comfortable bed located in a biophilic and restorative bedroom, as shown in FIG. 5, and the quality sleep that improves mental and cardiovascular health and immune functions. In another example, the database may include associations between a lounge chair located in a biophilic and restorative environment and the relaxation that supports rebalancing the autonomic nervous system and calming a person who experiences stress and anxiety symptoms.

As shown in FIG. 4, the health knowledge database may also include e-design information. For example, the e-design information may include AI-powered designs of biophilic spaces fostering evidence-based self-care practices listed in FIG. 1.

### Household Goods Database

The household goods database serves to store product information for products related to creating biophilic spaces and practicing self-care. In the example shown in FIGS. 4-6, the household goods database is communicably coupled to the health education database to integrate the household goods with the evidence-based knowledge about the health benefits of the goods.

Examples of home goods important for creating biophilic and health-centric spaces include: (1) smart lighting systems that provide dimmable natural light, (2) furniture made of natural materials that minimize exposure to harmful chemicals, (3) rugs made of natural materials for enhancing visual and haptic experiences in spaces that foster relaxation, (4) planters for indoor plants to reduce stress and improve positive mood, (5) air quality equipment including air quality monitoring systems and air purifiers to mitigate indoor air pollution. Comfortable clothing made from natural materials, already considered an interior design attribute, can also increase relaxation at home.

As can be seen in FIG. 5, the functionality of these consumer goods can be expanded by using them for daily communications with household occupants to empower their health behavior changes. For example, home goods that create a restorative refuge space for stress relief, or a restorative bedroom environment with embedded cues for sleep hygiene, can provide personalized updates on their health benefits via application programming interfaces (APIs) that integrate geo-positioning and object recognition applications, or applications working with machine-readable codes (e.g., visual and audible/inaudible acoustic QR codes).

### Consumer Goods Database

The consumer goods database serves to store product information for health-focused goods. In the example shown in FIG. 4, the health-focused consumer goods may include exercise and fitness goods, personal care goods, household care goods, supplements, electronic devices, and D2C diagnostic tests. The household goods database is communicably coupled to the health education database to integrate the consumer goods with the evidence-based knowledge about the product's health benefits.

Examples of diverse categories of consumer goods that can impact health outcomes are: (1) exercise and fitness equipment, (2) personal and household care products that minimize exposure to hazardous chemicals, (3) supplements that produce clinically meaningful effects, e.g. St John's Worth for depression, (4) direct-to-consumer diagnostic tests, educational materials supporting evidence-based self-care, (5) personal care and household care items that offer reduced exposure of household residents to health-harming chemicals, and (6) essential oils such as lavender or citrus essential oils for aromatherapy. The delivery of goods and services via the e-commerce platform 100 may help with creating a therapeutic home environment and sustaining healthy lifestyles.

### Consumer Database

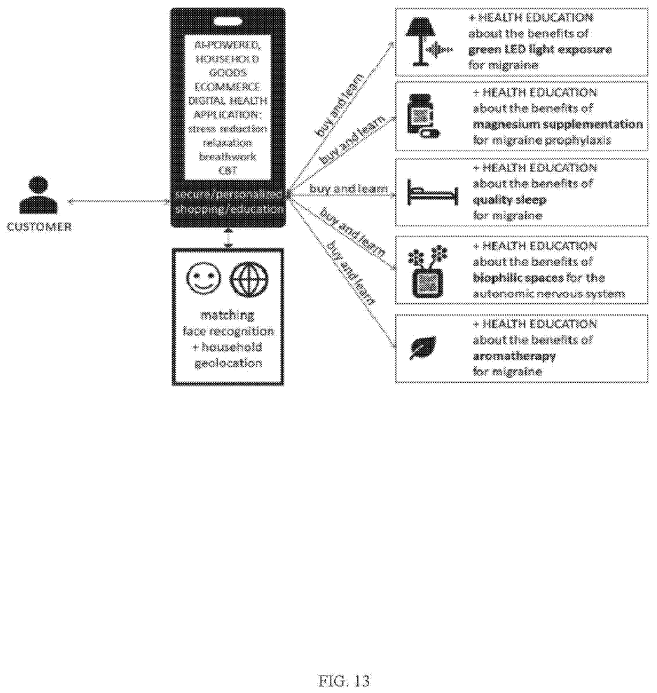

The consumer database serves to store the consumer's health information and information from the APIs relating to the purchase and use of the goods. The consumer database is communicably coupled to the health knowledge database, the household goods database, and the consumer goods database to personalize the consumer's shopping experience. In the example shown in FIG. 13, for people living with migraine, household items include information about the associations of specific household items, relaxation, and their abilities to reduce migraine headaches (green LED lamps, lavender essential oil, and aromatherapy diffuser, yoga mat, window curtains for controlling direct light coming through windows). In another example, for people living with chronic pain, household items include information about their abilities to reduce pain (audio speakers that deliver music, stationary bikes that deliver physical exercise).

### Application Programming Interfaces

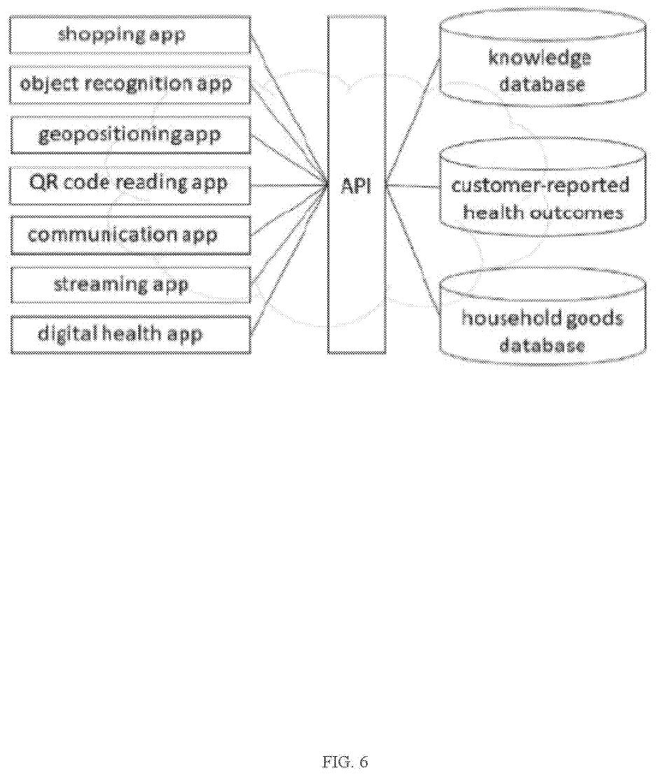

Application Programming Interfaces (APIs) serve as an intermediary that allows different applications to communicate with each other. APIs are a set of rules and protocols that define how applications can request and respond to each other. In the example shown in FIG. 6, an example of API infrastructure that integrates a home-centric e-commerce platform with personalized health education is illustrated. The APIs are communicably coupled to the health knowledge database, the household goods database, and the consumer goods database.

A visual or acoustic delivery of health education can also be accomplished using consumer electronics, such as mobile devices or smart TVs, which are the delivery system for biophilic, multisensory interventions. While applications of QR codes for health promotion and education exist, the intention to treat and prevent chronic diseases by using API communications, geolocation, and image recognition technologies within a home environment is a novel therapeutic strategy. The household occupants' awareness about their instant access to evidence-based knowledge and personalized updates about their health-centric home environment also serves as an empowerment tool to sustain behavior changes and exert therapeutic effects.

The health-centric, personalized communications accomplished using the APIs connect the household goods database with the knowledge database. Once the purchased good is delivered to a specific address of a customer's household, this good is positioned at home to maximize its use and relevant health outcomes. For example, when the customer purchased a bed, this good is located within a biophilic space as was illustrated using an AI-powered interior e-design service also integrated within the ecommerce platform, as shown in FIGS. 4-5.

The customer starts receiving the health benefits of using this bed by: (1) improving quality sleep because of restorative properties of a biophilic bedroom, and (2) continuous and personalized health education about the health benefits of sleep hygiene, biophilic design and quality sleep. For example, the customer experiences improved positive mood, reduction of anxiety symptoms, improved cardiovascular and immune functions, reduction of chronic pain, and migraine headaches. Referring to FIG. 5, continuous and personalized delivery of health-centric information about specific household items and a biophilic home environment is delivered using a communication app that is integrated with an object recognition app and a geo-positioning app.

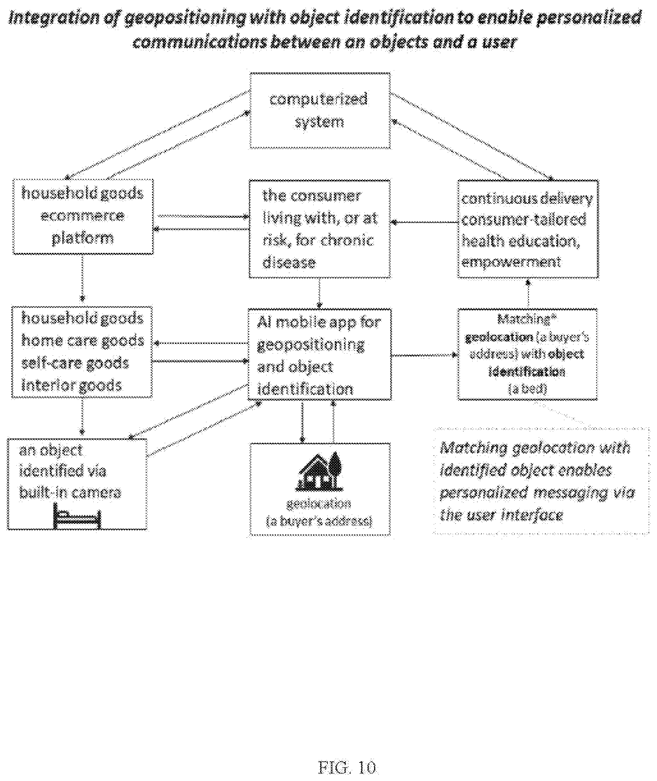

As can be seen in FIG. 10, the e-commerce platform 100 enables personalized communications between a household item and a household resident (the consumer) via the APIs. This system integrates an object recognition app that identifies a household item with a geo-positioning app that matches the identified household good with a consumer's address. This match ensures that health-centric communications are tailored to a household resident who lives with a specific chronic disease. For example, if a consumer lives with chronic pain, personalized communications between a bed and a consumer include information about how quality sleep contributes to the reduction and prevention of chronic pain. In another example, if a consumer lives with anxiety, personalized communications between a bed and a customer include updates on research studies about how quality sleep contributes to the reduction of anxiety symptoms and improvement of mental health. Personalized, health-centric communications are delivered on demand each time a consumer points a built-in camera in a mobile device or augmented reality glasses towards the household item that matches specific geolocation. The awareness about accessibility of health-centric information about household goods and home environment provides additional empowerment for the customer living with a specific chronic disease.

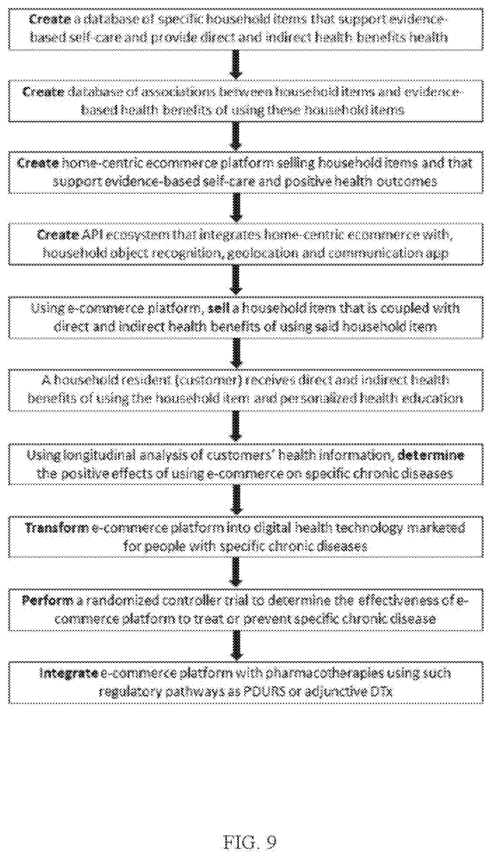

Turning now to FIG. 9, the effects of using the e-commerce platform 100 on specific health outcomes are determined, such as reduction of depressive or anxiety symptoms, decrease of chronic pain or migraine headache days, improvement of blood pressure, sleep, or cognitive functions. The changes in health conditions are obtained using patient-reported outcomes, biofeedback-based wearable devices, electronic health records, digital biomarkers collected using mobile devices, and other methods used in clinical studies to determine the efficacy and effectiveness of therapeutic interventions.

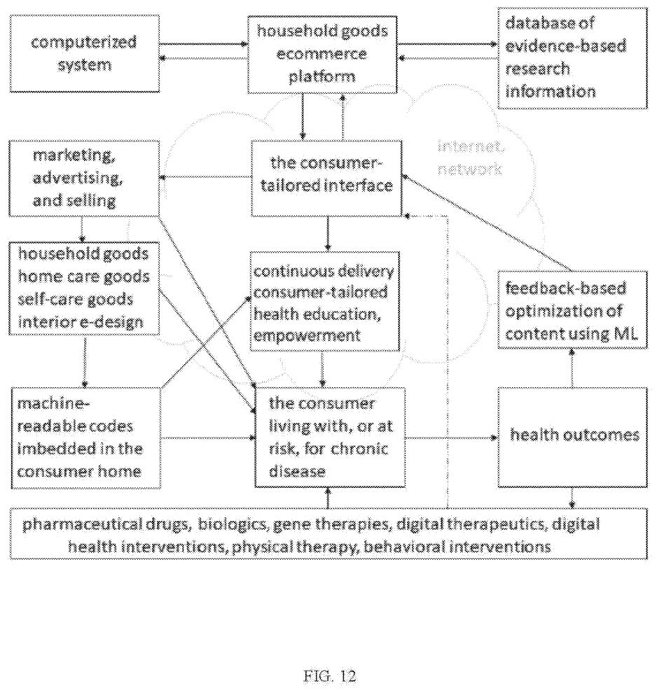

Continuous and personalized delivery of health-centric information about specific household items and biophilic home environment are delivered using a communication app that is integrated with object recognition app and geo-positioning app, as shown in FIG. 10, or using machine-readable codes, as shown in FIG. 12. The customer uses a built-in camera in a mobile device or an augmented reality glasses to deliver personalized information about the health benefits of household items located in the customer's home.

Machine-readable codes include visual QR codes, including dynamic QR codes that are embedded into household items, as well as audible/inaudible acoustic QR codes that are created using diverse electronic devices. To access information about the health benefits of specific household items, the customer uses a communication app that also recognizes machine-readable codes. Referring to FIG. 6, personalized, health-centric information is delivered via an API ecosystem that integrates all apps.

### Consumer Interfaces

The consumer interfaces provide the consumer access and interaction with the e-commerce platform. Some examples of consumer interfaces are a website or a mobile software application, as can be seen in FIG. 13.

### Additional Embodiments

The discussion will now focus on additional e-commerce platform embodiments. The additional embodiments include many similar or identical features to e-commerce platform 100. Thus, for the sake of brevity, each feature of the additional embodiment below will not be redundantly explained. Rather, key distinctions between the additional embodiments and the e-commerce platform 100 will be described in detail, and the reader should reference the discussion above for features substantially similar between the different e-commerce platform examples.

### Second Embodiment

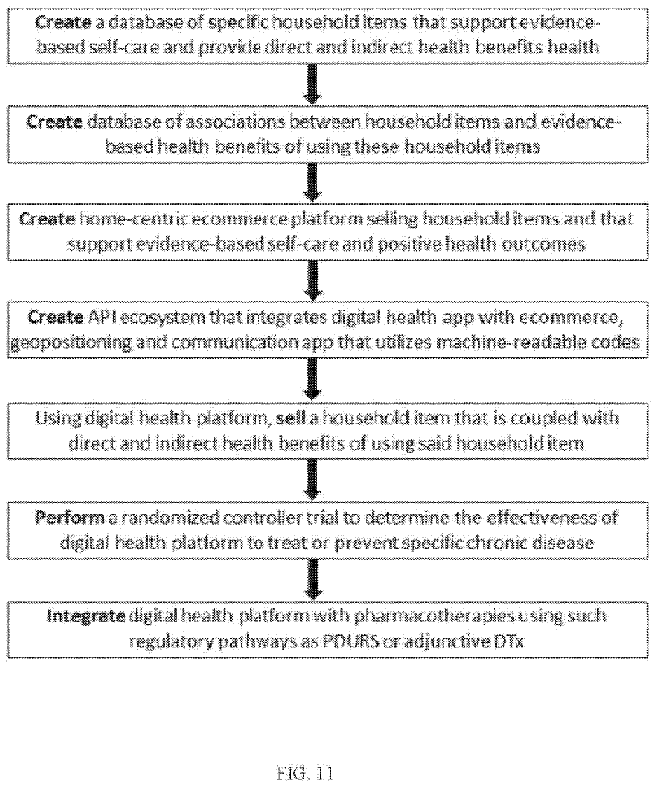

Turning attention to FIG. 11, a second example of an e-commerce platform, e-commerce platform 200, will now be described. As can be seen in FIG. 11, e-commerce platform 200 integrates an existing digital health platform intended to improve mental health with the e-commerce platform to further improve the effectiveness of digital intervention. This integration enables the user to improve engagement and health outcomes of the customer who more often uses the integrated digital health ecommerce platform for both shopping needs and improvement of mental health, while receiving empowerment intervention through continuous health education within the customer's home environment.

### Third Embodiment

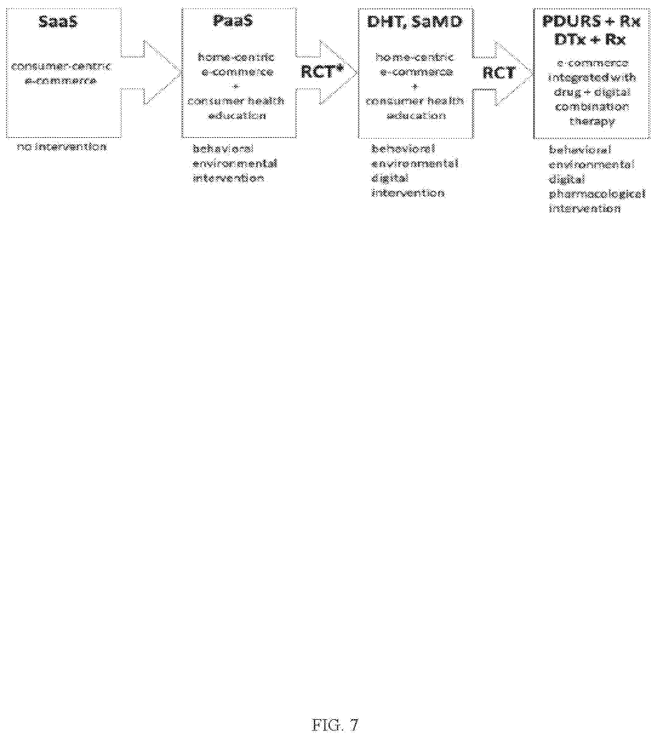

As also shown in FIG. 7, a third example of an e-commerce platform, e-commerce platform 300, will now be described. E-commerce platform 300 is an existing e-commerce platform integrated with therapeutic content (shown as SaaS). A distinction between e-commerce platform 300 and e-commerce platform 100 is that e-commerce platform 300 utilizes an existing e-commerce platform, whereas e-commerce platform 100 is a health-centric e-commerce platform.

### Fourth Embodiment

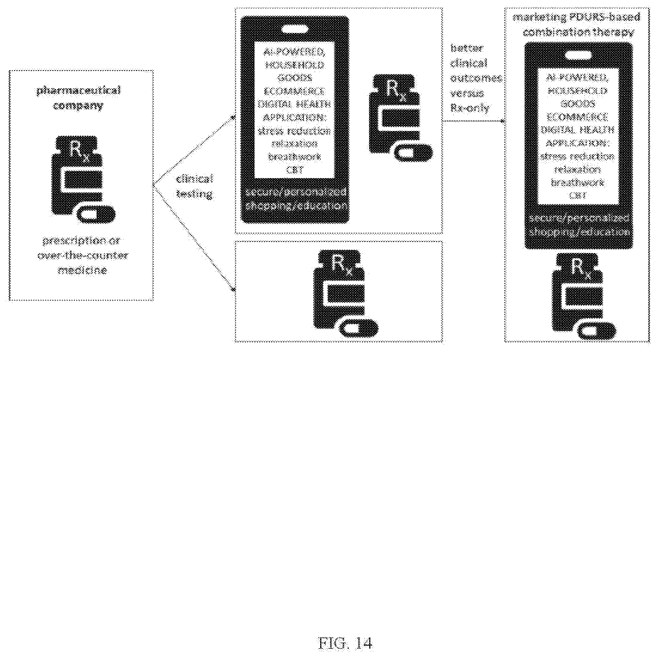

Turning to FIGS. 13 and 14, a fourth example of an e-commerce platform, e-commerce platform 400, will now be described. As can be seen in FIG. 14, e-commerce platform 400 is an e-commerce platform integrated with a drug and digital combination therapy (shown as PDURS+Rx or DTx+Rx). A pharmaceutical company improves the success rate for developing a pharmaceutical drug and biologic for the treatment of chronic pain by clinically testing an investigational new drug (IND candidate) in the absence and presence of a household goods ecommerce platform, exemplified in FIG. 6. The household goods ecommerce platform provides both (1) items that support patients' quality sleep, physical exercises at home, mindfulness meditation, aromatherapy, and anti-inflammatory dietary intervention, along with (2) personalized health education about research studies on clinical benefits of self-care practices supported by the aforementioned items. The use of the household goods ecommerce platform as an adjunctive treatment is accomplished by either (1) using the household goods ecommerce platform alone, or (2) using the household goods ecommerce platform integrated with a digital health app delivering therapeutic content intended to treat chronic pain conditions, such as physical therapy or cognitive behavioral therapy. The patient treated by a combination of the investigational new drug and the household goods ecommerce platform has better long-term pain management outcomes, including clinically meaningful improvements in pain relief Upon review of results from a randomized controlled trial, the pharmaceutical company receives a regulatory authorization to label and market the pain medication together with the household goods ecommerce platform using a “prescription drug use-related software” framework.

Turning now to FIG. 7, the use of the e-commerce platform 400 in combination with pharmacotherapies (pharmaceutical drugs, biologics), gene therapies, and other digital health interventions determines clinically meaningful benefits. These benefits enable marketing of the household goods ecommerce platform as: (1) adjunctive digital therapeutics in combination with a specific class of drugs, (2) “prescription drug use-related software” in combination with a specific drug, and (3) a drug-device combination product in which a specific drug is integrated with an e-commerce platform that has a status of medical device. These are examples of drug+digital combination therapies in which a prescription drug or an over-the-counter drug is used in combination with the household goods ecommerce platform targeting the home environment for treatment and prevention of specific chronic medical conditions.

### Illustrative Example—Reduction in Monthly Migraine Days

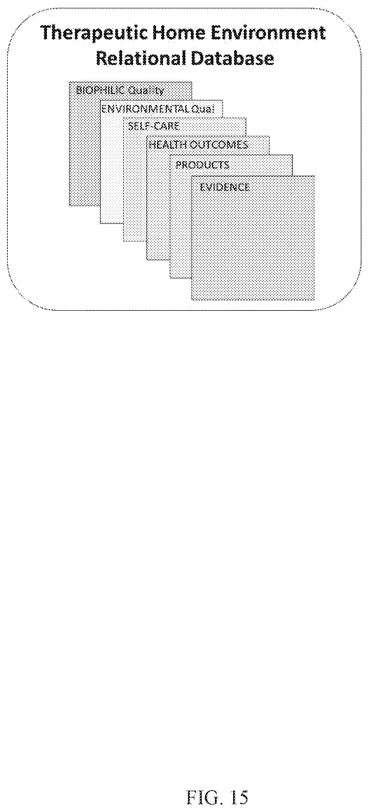

FIG. 15 is a schematic of the Therapeutic Home Environment relational database, illustrating interconnected tables—biophilic quality, environmental quality, self-care, health outcomes, products, and evidence—that collectively generate personalized, evidence-based home optimization recommendations. The Therapeutic Home Environment (THE) database is a relational, evidence-indexed data architecture that enables the mobile app to generate precise, science-based recommendations for optimizing the user's living space to provide therapeutic effects. Each table represents a core domain of the therapeutic home model—Biophilic Quality, Environmental Quality, Self-Care, Health Outcomes, Products, and Evidence—and each of these data layers is linked through well-defined foreign keys and relational mappings. This structure allows the system to dynamically connect environmental features and user behaviors to validated health mechanisms and product-level interventions.

Biophilic Quality and Environmental Quality tables store structured environmental attributes (e.g., natural materials, lighting spectra, air-quality metrics, acoustic properties). These attributes are cross-linked to the Self-Care table, which defines behavioral routines affected by environmental context (sleep hygiene, relaxation practices, stress regulation, physical activity). The Health Outcomes table contains condition-specific endpoints-such as migraine frequency, stress reduction markers, sleep improvements, and pain reduction-mapped to both environmental variables and self-care behaviors. Product entries in the Products table are tagged with environmental and biophilic attributes (e.g., “low-blue light lamp,” “HEPA filtration,” “green-spectrum LED lamp,” “plant species with high VOC removal”), enabling the system to connect each product to specific therapeutic mechanisms.

The Evidence table functions as the scientific backbone of the system. Each evidence entry (e.g., peer-reviewed study, mechanistic insight, effect size estimate) is linked to environmental attributes, behavioral interventions, and health outcomes. When a user completes assessments, the app queries these relational tables to identify which environmental deficits exist, which self-care competencies are most affected, which health outcomes are modifiable, and which products are scientifically supported to address them. This relational approach allows the app to deliver personalized, evidence-aligned household product recommendations that directly support the creation of a Therapeutic Home Environment.

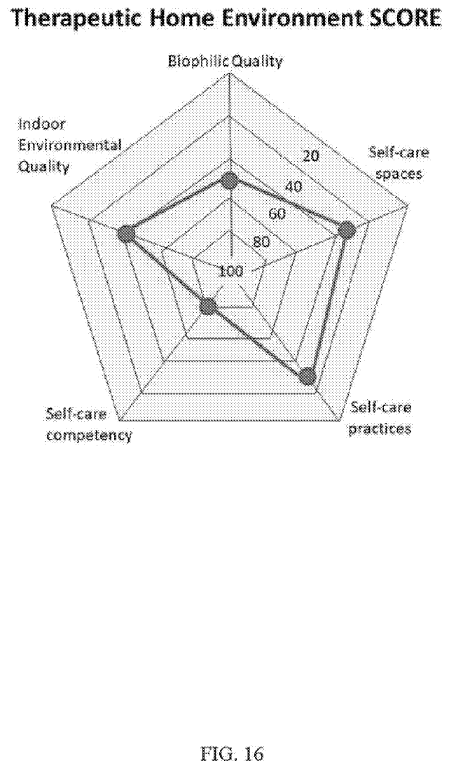

FIG. 16 is a radar chart illustrating the Therapeutic Home Environment Score across five domains—biophilic quality, indoor environmental quality, self-care spaces, self-care practices, and self-care competency—to visualize strengths and deficits within the user's home environment. It illustrates the Therapeutic Home Environment (THE) Score, a multidimensional metric used in the mobile app to quantify how effectively a user's household supports migraine regulation, emotional balance, sleep quality, and overall neurological health. The radar chart visualizes performance across five core domains-Biophilic Quality, Indoor Environmental Quality, Self-care Spaces, Self-care Practices, and Self-care Competency-allowing users to quickly identify strengths and deficits within their home ecosystem. Within the app, this score is recalculated dynamically as the user completes assessments, updates the wellness diary, and adopts recommended environmental or behavioral changes.

Each domain represents a distinct pillar of the therapeutic home environment intervention. Biophilic Quality captures the degree of natural elements, materials, and restorative sensory inputs within the home; Indoor Environmental Quality assesses air quality, pollutant load, acoustics, and lighting spectra; Self-care Spaces evaluates whether the home contains intentional areas designed to support relaxation, recovery, and health-promoting routines; and Self-care Practices measures adherence to daily behaviors such as sleep hygiene, stress management, and physical activity. By visualizing these domains together, the app helps the user understand how environmental factors interact with daily self-care behaviors to influence symptom patterns.

The self-care competency includes self-efficacy, self-compassion, and self-regulation. Self-efficacy is the patient's belief and confidence in their own ability to heal, as determined by their knowledge, experiences, motivation, and expectations. Higher self-efficacy can improve headache-related disability and adherence to behavioral therapies. Self-compassion (positive attitude towards the self) and self-regulation (the ability to manage thoughts, emotions, and actions) are connected and vital for health outcomes. For example, self-compassion supports health behaviors and is associated with better physical health, sleep, and emotional regulation. Self-regulation encompasses self-monitoring, feedback, and goal setting to improve health behavior changes and sustained therapy outcomes. Cognitive behavioral therapy (CBT) can improve self-efficacy, self-compassion, and self-regulation.

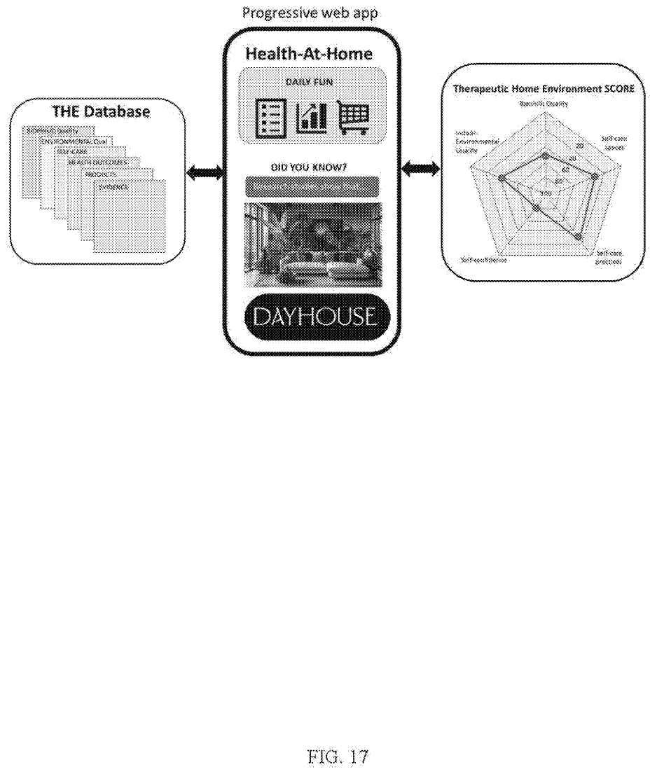

FIG. 17 is an example of an app that enables the user to reduce migraine symptoms through a household goods e-commerce platform that communicates with (1) the therapeutic home environment database and (2) embedded analytics to compute a quantified Therapeutic Home Environment (THE) score from user-specific inputs. The score is calculated based on biophilic, environmental quality, and self-care variables This closed-loop data-analytics-feedback system supports personalized optimization of the therapeutic home environment, and thus improved therapy outcomes.

As illustrated in FIG. 17, the progressive web app (PWA) delivers an integrated, evidence-based platform designed to evaluate, optimize, and monitor the user's Therapeutic Home Environment (THE) in relation to migraine management and self-care behaviors. The onboarding sequence functions as a structured data-acquisition pipeline, capturing demographic variables, symptom burden, environmental constraints, behavioral baselines, and user goals. Users then complete both an initial screening assessment and a more granular multidomain environmental audit, which quantifies home-environment factors across lighting, air quality, acoustics, spatial organization, materials, nature exposure, and self-care micro-routines. These data feed into the app's scoring engine, generating a THE Score and personalized environmental risk-opportunity profile.

The platform's core functionality centers on a dynamic THE Optimization Engine, powered by a proprietary, science-based database that links environmental features with psychological, physiological, and neurological mechanisms relevant to migraine. Users receive targeted recommendations ranked by predicted effect size, feasibility, and cost. These recommendations connect directly to an integrated e-commerce module, offering vetted household products-lighting, textiles, air-quality devices, organizational tools, and biophilic elements-mapped to the specific deficits detected in the assessment. A daily education feed uses push-based micro-lessons to reinforce the connection between home environment, self-care practices, and symptom regulation.

A dedicated migraine journaling, monitoring, and tracking system enables the user to log triggers, exposures, symptoms, medications, routines, and environmental adjustments. The system cross-references journal entries with home-environment changes to model longitudinal trends and provide feedback loops for behavior-environment interactions. Over time, users visualize their progress through THE Score trajectory charts, stability metrics, and personalized insights that reveal which environmental and self-care interventions deliver the strongest clinical or functional impact for that individual.

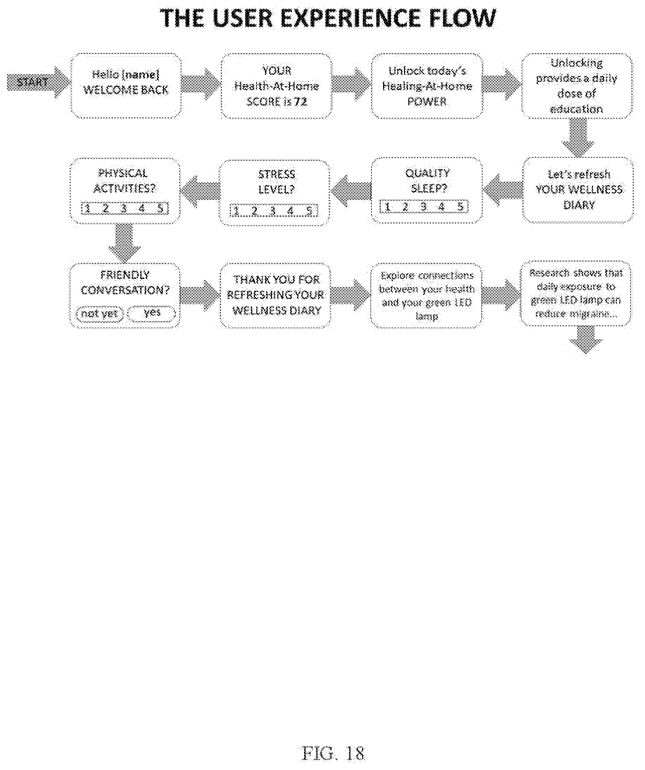

FIG. 18 is a user experience flow diagram illustrating the daily interaction sequence in the mobile app, including personalized greeting, therapeutic home environment score display, educational micro-intervention, wellness diary inputs, and tailored therapeutic guidance. The user experience flow in the mobile app is structured as a daily, closed-loop behavioral and environmental monitoring cycle that integrates assessment, education, and reinforcement within the Therapeutic Home Environment (THE) intervention. The session begins with a personalized re-engagement module, greeting the user by name and immediately presenting their current Health-At-Home Score, a composite metric derived from environmental inputs, behavioral logs, and prior THE optimization actions. This primes the user with a clear status indicator before moving them into the “unlock” phase, which gates that day's micro-intervention or educational insight. Unlocking the daily module triggers delivery of a small, digestible educational asset-typically a research-backed insight about home environment, self-care, or migraine regulation-designed to drive micro-learning and contextual relevance.

After the educational element is delivered, the user is transitioned into the Wellness Diary sequence, a structured micro-assessment with three primary behavioral domains: sleep quality, stress level, and physical activity. Each variable is rated through a low-friction 1-5 input interface, optimized for daily adherence. Inputs update the app's internal state model and recalibrate the Health-At-Home Score. Following these entries, the system optionally prompts a light-weight social/reflective interaction (“Friendly Conversation?”), which acts as an engagement checkpoint or journaling catalyst. Once the diary is completed, the app surfaces personalized, context-aware feedback, such as evidence-based explanations linking user behaviors or environmental exposures (e.g., green LED light therapy) to migraine outcomes. This closes the loop by reinforcing the value of both data entry and environmental optimization, encouraging continued daily interaction, and supporting the longitudinal therapeutic impact of the intervention described in this invention.

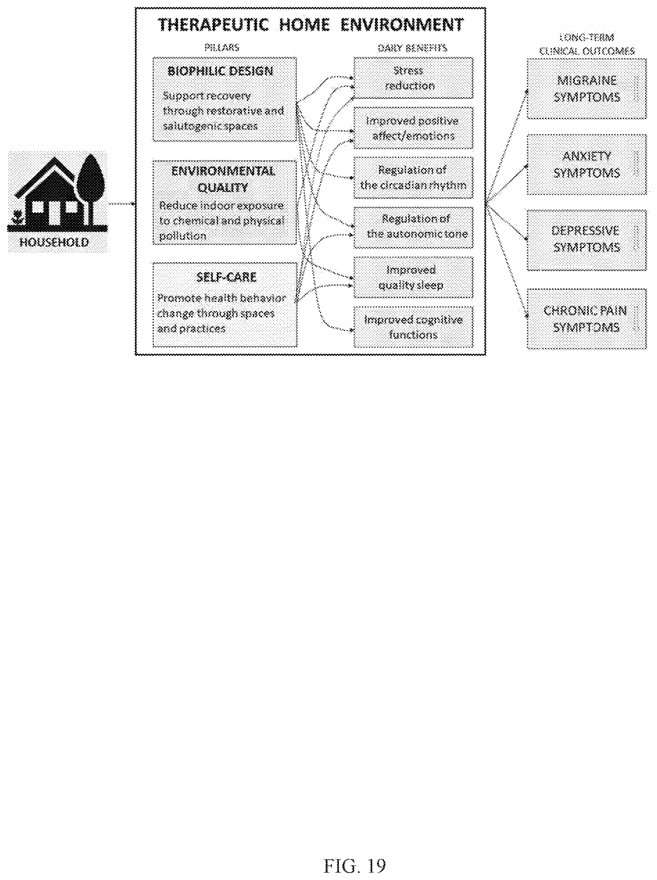

FIG. 19 illustrates converting a household into a therapeutic asset using the three pillars of a Therapeutic Home Environment—biophilic design, environmental quality, and self-care. The diagram depicts the Therapeutic Home Environment as a multimodal, adjunctive intervention in which biophilic design, indoor environmental quality, and self-care-supportive spaces act as modifiable household-level variables that influence neurophysiological and psychological processes relevant to treatments for neurological and mental disorders. Each pillar targets a specific mechanism: biophilic design leverages restorative and salutogenic exposure; environmental quality reduces chemical and physical indoor pollutants that drive allostatic and neuroinflammatory load; and self-care-oriented spatial design increases adherence to health behaviors that modulate autonomic regulation, sleep, and emotional regulation.

These pillars converge to produce daily, measurable benefits-stress reduction, improved affect, circadian regulation, autonomic balance, higher-quality sleep, and enhanced cognitive function. Over time, these upstream effects influence clinical outcomes associated with migraine, anxiety, depression, and chronic pain, resulting in reduced symptom severity and improved long-term therapy outcomes. The invention illustrates the therapeutic home environment (delivered through the household goods ecommerce as a digital health platform) as a continuous, environment-behavior-physiology feedback system in which optimized home conditions modulate neurobiological effects and support multimodal treatment of neurological and mental health disorders.

The main goal of migraine prevention is reaching either: (a) “optimal control” (defined as less than four monthly migraine days (MMD) for three consecutive months), (b) “migraine freedom” (no MMD for three consecutive months), or (c) “total freedom from headaches and associated symptoms”. However, reaching optimal control of migraine freedom for people living with chronic migraine (>15 MMD) or refractory migraine (defined as more than eight migraine episodes per month despite trying all available treatment options) is challenging, even when reaching more than 50% reduction of MMD through prescription drugs. It is apparent that developing a cure for migraine requires a personalized and multimodal strategy that leads to and supports a sustained freedom from all symptoms and comorbidities. Table 1 shows data from randomized controlled trials of FDA-approved treatments for migraine. These data show that such treatments can reduce one to four MMD, a therapeutic effect that is not sufficient for the user who experiences more than 4 MMD. These limitations of the effectiveness of currently available FDA-approved treatments highlight opportunities to improve migraine treatment and prophylaxis by combining the present invention with pharmacological and medical device-based interventions.

Table 1 below provides examples of FDA-approved treatments for migraine prophylaxis that can reduce monthly migraine days (MMD) in episodic and chronic migraine. MMD data are based on meta-analysis or RCT studies and are not placebo-corrected. These data illustrate limitations of currently available medical treatments, as migraine patients experiencing five or more monthly migraine days often have difficulty achieving migraine freedom with the treatments listed in Table 1

TABLE 1

| | MMD | |
|---|---|---|
| Intervention | Reduction | Mechanism of Action |
| Rimegepant | 3.6 | CGRP receptor antagonist |
| Frenamezumab | 2.2 | Anti-CGRP monoclonal antibody |
| Botox | 2 | Modulation of neurotransmitter release |
| Topiramate | 1 | ASM modulation of VGCC and |
| | | GABA-mediated inhibition |
| Propranolol | 1.5 | Non-selective beta-blocker |
| Nerivio | 4 | Remote electrical neuromodulation of |
| | | the conditioned pain pathway |
| DTx CT-132 | 3 | Cognitive/behavioral intervention |
| (+Rx) | | targeting patient responses to |
| | | environmental and internal stimuli |

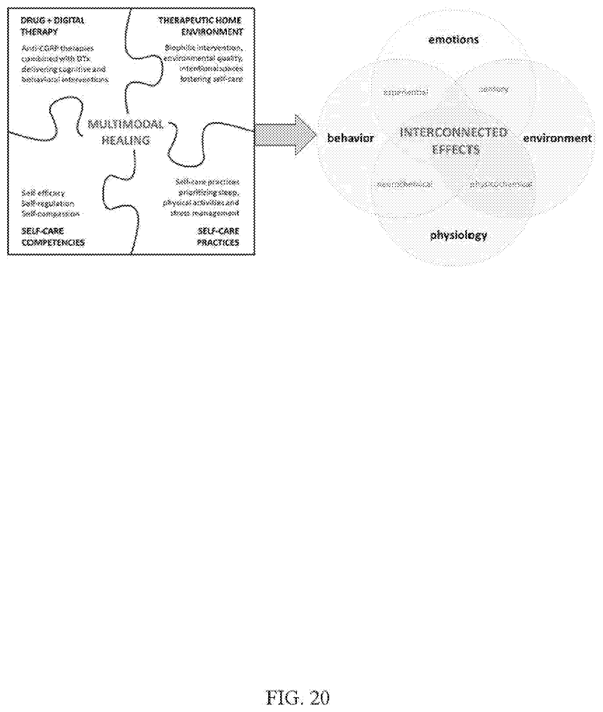

FIG. 20 is an example of a personalized, multimodal cure for migraine that integrates the drug+digital combination therapy with the therapeutic home environment and self-care practices. Daily delivery of diverse modalities and continuous engagement elicits interconnected therapeutic effects. The present invention describes a household goods e-commerce platform that delivers the Therapeutic Home Environment as an adjunctive treatment. This treatment modifies environmental, sensory, and behavioral inputs that interact with the user's migraine-relevant neurobiological pathways. As illustrated in the FIG. 20, the present invention enables the delivery of the therapeutic home environment intervention that is integrated with pharmacologic and digital therapies within a multimodal therapy. The multimodal therapy targets several mechanistic domains (sensory, physicochemical, experiential, neurochemical), reducing allostatic load and stabilizing physiological systems implicated in migraine, including cortical excitability, trigeminovascular activation, and dysregulated stress-response circuits. In this intervention, the user's home environment becomes a continuous therapeutic interface that significantly reduces monthly migraine days. Because environment, emotion, physiology, and behavior are interconnected systems, the present invention enhances the effectiveness of medications and digital therapies. When implemented as part of an integrated care pathway, the invention reduces environmental triggers, reinforces behavioral change, and creates conditions that make therapeutic routines (sleep hygiene, exercise, stress management) more achievable and sustainable. This synergistic effect positions the home as an active therapeutic ecosystem.

Applicant(s) reserve the right to submit claims directed to combinations and subcombinations of the disclosed inventions that are believed to be novel and non-obvious. Inventions embodied in other combinations and subcombinations of features, functions, elements, and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

1. A method for improving outcomes of medical interventions by using an integrated e-commerce platform, where the integrated e-commerce platform comprises the following proprietary platforms and databases: an e-commerce platform, a health knowledge database, a household goods database, a consumer goods database, a consumer reports database, an application programming interface, and consumer interfaces.

2. The integrated e-commerce platform of claim 1 further comprises an existing digital health platform integrated with the integrated e-commerce platform.

3. The integrated e-commerce platform of claim 1, wherein the integrated e-commerce platform further comprises an existing e-commerce platform.

4. The integrated e-commerce platform of claim 1, where the integrated e-commerce platform is integrated with a drug and digital combination therapy

5. A method for improving outcomes of medical interventions by creating a therapeutic home environment by using an integrated e-commerce platform of claim 1 to evaluate home biophilic and environmental quality, user self-care, and household items.

6. A method for evaluating the quality of a therapeutic home environment by using an integrated e-commerce platform of claim 1 to create a therapeutic home environment score.

7. A method to improve interventions for chronic diseases comprising: using a household goods e-commerce platform to continuously deliver personalized educational messages to consumers in order to support health-centric behavior changes using an application programming interface, geopositioning, object detection and identification, and machine-readable codes integrated within a home environment through said household goods.

8. A method to improve interventions for chronic diseases comprising: using e-commerce to deliver household goods that use visual and acoustic machine-readable codes to provide information about evidence-based health benefits of using said household goods at home.

9. A method to improve interventions for chronic diseases comprising: using household goods e-commerce that utilizes machine learning and artificial intelligence to optimize personalized information and cues for health-centric behavior changes using mobile devices, augmented reality glasses, geo-positioning, object identification, and machine-readable codes integrated within a home environment.

10. The method of claim 7 comprising: a computerized system, application programming interface, cloud computing, mobile electronic devices, augmented reality glasses, wearables providing biofeedback, visual and acoustic machine-readable codes, communication app, geolocation app, object identification app, web-based app, user interface furniture and home décor, rugs, curtains, fitness and wellness equipment, indoor lighting systems, lamps, consumer electronics, home electronics, outdoor patio furniture, outdoor patio décor, gardening tools, indoor air purifier, water purification system, indoor water features, personal care goods, dietary supplements, nonperishable foods, household care goods, planters, indoor plants, audio and visual electronic systems, essential oil diffusers, clothing, tableware.

11. The method of claim 7, wherein,
chronic diseases include at least one of the following:
(a) migraine, chronic pain, arthritis, depression, anxiety, cancer, Alzheimer's disease, dementia, diabetes, cardiovascular diseases, autoimmune disease, and Parkinson's disease;
(b) where intervention, for causes and symptoms of aforementioned chronic diseases, includes at least one of the following medical treatments including pharmacotherapy, gene therapy, digital therapy, digital therapeutics, prescription drug use-related software application, physical therapy, cognitive behavioral therapy, primary prevention, secondary prevention, tertiary prevention.

12. The method of claim 7, wherein,
consumers and patients use their own mobile and wearable devices to provide biofeedback-based, health outcomes in order to optimize personalized information and cues for health-centric behavior changes using machine-readable codes integrated into a home environment consumers and patients use their own electronic health records to optimize personalized information and cues for health-centric behavior changes using machine-readable codes integrated into a home environment consumers and patients use their own augmented reality glasses and mobile devices to receive personalized health education enabled by object identification and geolocation apps.

13. A computerized system to improve therapeutic interventions for chronic diseases comprising: using a household goods e-commerce platform to deliver personalized messages supporting health-centric behavior changes using mobile devices, augmented reality glasses, geo-positioning, object identification, and machine-readable codes integrated into a home environment.

14. A computerized system to improve interventions for chronic diseases comprising: using a digital health application integrated with an e-commerce marketplace platform to deliver household goods supporting evidence-based self-care practices and health education about the clinical benefits of using said household goods and self-care practices at home.

15. A computerized system to improve interventions for chronic diseases comprising: using a household goods e-commerce marketplace and interior design that utilizes machine learning and artificial intelligence to optimize personalized information for health-centric self-care and behavior changes using object identification and machine-readable codes integrated into a home environment.

16. The system of claim 7 comprising:
a cloud computing, mobile electronic devices, augmented reality glasses, wearables providing biofeedback, visual and acoustic machine-readable codes, application processing interface, mobile app, geo-positioning app, object identification app, communication app, web-based app, and user interface.

17. The computerized system of claim 16, wherein,
chronic diseases include at least one of the following:
(a) migraine, chronic pain, arthritis, depression, anxiety, cancer, Alzheimer's disease, diabetes, cardiovascular diseases, and Parkinson's disease;
(b) where disease intervention, for the aforementioned chronic diseases, includes at least one of the following: medical treatments of causes and symptoms of aforementioned chronic diseases, pharmacotherapy, gene therapy, digital therapy, digital therapeutics, prescription drug use-related software application, physical therapy, cognitive behavioral therapy, primary prevention, secondary prevention, tertiary prevention.
